(19) United States
(12) Patent Application Publication
(10) Pub. No.: US 2021/0236341 A1
(43) Pub. Date: Aug. 5, 2021

(54) PROTECTIVE DRESSING FOR ABDOMINAL VISCERA IN NEGATIVE PRESSURE THERAPY

(71) Applicant: SERVICIO CANTABRO DE SALUD, Santander (ES)

(72) Inventor: Federico Castillo Suescun, Santander (ES)

(73) Assignee: SERVICIO CANTABRO DE SALUD, Santander (ES)

(21) Appl. No.: 16/972,964

(22) PCT Filed: Jun. 3, 2019
(86) PCT No.: PCT/ES2019/070372

§ 371 Date: Dec. 7, 2020

(30) Foreign Application Priority Data
Jun. 6, 2018 (ES) U201830853

(51) Int. Cl. A61F 13/00; A61F 13/14

(52) U.S. Cl. CPC A61F 13/00068; A61F 13/00029; A61F 13/14

(57) ABSTRACT

Protective dressing for abdominal viscera for application of negative pressure therapy in open abdomen, made up of an upper layer (1), an intermediate layer (3) and a lower layer (2), each of which consists of a laminar body. The upper layer (1) includes a plurality of first holes (6) for the passage of fluid and the intermediate layer (3) includes numerous low pressure gas filled bubbles (7) and a plurality of second holes (8) for the passage of fluid. These bubbles (7) prevent possible damage to the abdomen and prevent the sheets from sticking together. The three layers adhere to each other at their respective perimeter edges.

### OBJECT OF THE INVENTION

The present invention falls within the technical field of bandages or dressings, as well as that of devices for draining wounds or the like, and relates in particular to a protective dressing for abdominal viscera in open abdomen for use in negative pressure therapy.

### BACKGROUND OF THE INVENTION

In the cases of surgical operations inside the abdominal cavity that require making transcutaneous incisions to access said interior, the primary closure of the abdominal cavity restores the anatomy and physiology of the abdominal wall, providing protection to the abdominal content and reducing the complications of the surgical wound. However, there are certain circumstances, such as abdominal trauma, severe acute pancreatitis, severe septic abdomen or Abdominal Compartment Syndrome, wherein primary closure is not possible, making it necessary to establish a system for wound management that facilitates reentry into the abdominal cavity and allow successive operations.

Forced closure or tension of the cavity can cause increased intra-abdominal pressure (IAP) in the patient and lead to the aforementioned Abdominal Compartment Syndrome (ACS), which is a condition that involves an increase in pressure in a compartment, in this case the abdominal cavity, which causes an anomalous venous return as well as a difficulty in the blood supply to the viscera, leading to hypo-perfusion and finally, with an increase in IAP, to ischemia due to a decrease in blood flow, arterial blood perfusion and venous congestion that can lead to the death of the patient.

Over the years, various strategies have been developed to try to mitigate intra-abdominal hypertension and prevent its progression to ACS. In addition to being necessary to facilitate reentry into the abdominal cavity, it may be desirable to remove fluids from the cavity and apply therapy that reduces pressure on the tissue or wound. This therapy, frequently referred to in the medical community as negative pressure therapy (NPT), provides a number of benefits: decreases wound retraction, removes exudate and non-viable tissue, improves blood supply, promotes granulation tissue generation, physically stimulates mitosis and, as a consequence of all this, facilitates healing and closure of the abdominal cavity.

Various devices for applying NPT as well as dressings and drainage components of said systems are known in the current state of the art. For example, the document under publication number WO2013034262 describes a dressing for negative pressure therapy comprising a flexible film, with a first and a second side, the first side being provided for application on the wound and, the flexible film comprising openings that are distributed over the entire surface.

Furthermore, said dressing comprises at least three sections of conduits, on the second side of said flexible film.

Although it is described that said film consists of a material that should have atraumatic properties, to prevent it from adhering to internal organs, said dressing generates a negative pressure on the mesothelium that passes through the dressing and, when withdrawn, produces a lifting of part of the tissue also leaving a mark on the tissues through the openings.

On the other hand, the patent application AU2016277595, describes a system for applying NPT that comprises a device for treatment with a plurality of members encapsulated and placed on a surface. Said members are coupled to a central connector. Fenestrations can be formed in the plurality of encapsulated members, or in the central connector, which allow fluids in the abdominal cavity to pass through. However, the encapsulated member quickly saturates and ceases to exert an effective negative pressure during therapy.

In view of the state of the art, there arises a need for devices that improve negative pressure therapy, facilitating the closure of the abdominal cavity and avoiding or minimizing complications, such as deep tissue retraction or necrosis.

### DESCRIPTION OF THE INVENTION

The object of the invention consists of a protective dressing for abdominal viscera for the application of negative pressure therapies in the open abdomen, which avoids the aforementioned drawbacks and also allows optimizing the distribution of negative pressure over the surface of the sheet to the most distal areas and improve drainage capacity.

To this end, said dressing comprises a plurality of laminar layers overlapped and integrally linked to each other by at least their respective perimeter edges. In its preferred embodiment, the dressing is made up of three layers: an upper layer, a lower layer and an intermediate layer, arranged between the upper and lower layers.

The upper and lower layers each consist of a laminar body with an internal side, an external side and a plurality of first through holes having reduced dimensions and uniformly distributed that allow the passage of fluids through them.

For its part, the intermediate layer consists of a laminar body that incorporates a plurality of alveoli or bubbles evenly distributed and filled with low pressure air. Low pressure air prevents possible damage to the abdomen. In a preferred embodiment, said bubbles have an essentially hemispherical geometry and are uniformly distributed throughout the intermediate layer.

It is also envisaged that said distribution may correspond to radial, concentric, spiral or barred patterns, as well as that the bubbles have a conical or cylindrical geometry. In alternative embodiments, the bubbles can be filled with CO₂ or other low-pressure gases.

This intermediate layer also incorporates a plurality of second through holes adjacent to the bubbles, to allow the passage of fluids through them, giving rise to an intermediate layer of a fenestrated nature. These second holes also allow the transmission of negative pressure to the abdominal cavity. In a preferred embodiment, the second through holes have an essentially rhomboid geometry.

The protective dressing for abdominal viscera thus described is temporarily placed on an open abdomen, in particular on the internal organs of the abdomen or on the greater omentum, indistinctly, on one of the external sides of the upper or lower layers. A spongy body is in turn arranged on the dressing, on which a negative pressure therapy system is supported.

By applying negative pressure to the dressing, the air-filled bubbles prevent the sheets from adhering to each other in the first place, as this configuration prevents rapid saturation and clogging of the polyurethane sponge contained in other dressings with more dense and solid elements like the remains of pus, fibrin clots and necrosis.

The bubbles also prevent adherence to the tissues of the internal organs of the abdominal wall, as well as the effluents from the internal organs (fluids, necrotic tissues, clots, etc.) drain homogeneously through the through holes. The uniform distribution of both the air bubbles and the through holes over the entire surface of the respective layers optimizes the drainage capacity and the distribution of negative pressure therapy, allowing it to be exercised even in the most distal areas of the cavity.

The dressing is preferably made of a material selected from the group of EVA, PU, PVC,

PE, PET, PTFE, TPE, silicone or a mixture thereof. Also, the dressing can be adjusted to the size of the opening of the abdomen, depending on the treatment required, cutting the necessary surface.

### DESCRIPTION OF THE DRAWINGS

To complement the description that is being made and in order to help a better understanding of the features of the invention, according to a preferred example of a practical embodiment thereof, a set of drawings is attached as an integral part of said description in which, for illustrative and non-limiting purposes, the following has been represented:

FIG. 1. Shows an exploded perspective view of the dressing, wherein its main constituent elements are seen.

FIG. 2. Shows a plan view of the dressing.

### PREFERRED EMBODIMENT OF THE INVENTION

A detailed explanation of a preferred embodiment of the object of the present invention is provided below, with the aid of the aforementioned figures.

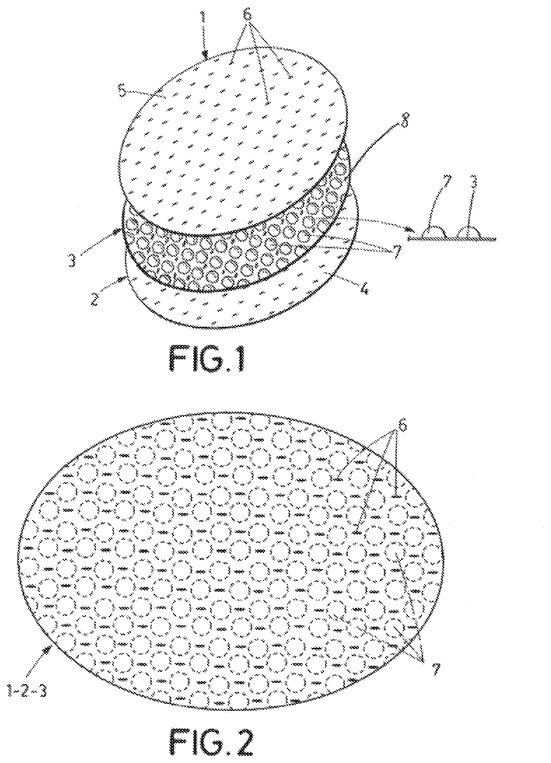

The protective dressing for abdominal viscera in negative pressure therapy that is described is made up of a multilayer structure, which in this preferred embodiment is comprised of an upper layer (1), a lower layer (2) and an intermediate layer (3) located between the upper (1) and lower (2) layers, the three layers being integrally linked to each other by their respective perimeter edges as seen in FIG. 1.

The upper (1) and lower (2) layers, having similar geometry and dimensions, each consist of a laminar body with an internal side (4), intended to be oriented inwards and facing the intermediate layer (3) and an external face (5), intended to be oriented towards the outside. Likewise, the laminar body incorporates a plurality of first evenly distributed through holes (6) that allow the passage of fluids through them, which gives said upper (1) and lower (2) layers a fenestrated nature.

The intermediate layer (3), having geometry and dimensions essentially similar to those of the upper (1) and lower (2) layers, consists of a laminar body that incorporates a plurality of bubbles (7) uniformly distributed and filled with a gaseous fluid at low pressure. In the preferred embodiment described herein, said bubbles (7) have an essentially hemispherical geometry, as illustrated in the detail in FIG. 1.

The intermediate layer (3) also incorporates a plurality of second through holes (8) adjacent to the bubbles (7) that allow the passage of fluids through them, giving rise to an intermediate layer (3) of a fenestrated nature, as well as allowing the transmission of negative pressure to the abdominal cavity. In this preferred embodiment, the second through holes (8) have an essentially rhomboid geometry. The separation distance between bubbles (7) in said intermediate layer (3) shall be at least half their diameter.

As can be seen in FIG. 2, in the preferred embodiment the overlapping of the upper (1), lower (2) and intermediate (3) layers is carried out in such a way that the respective through holes (6) are facing each other, without the bubbles (7) being facing said through holes (6).

In an alternative embodiment, not shown in the attached figures, the multilayer structure of the protective dressing consists only of the upper layer (1) and the intermediate layer (3), which is directly facing the abdominal cavity, regardless of the lower layer (2). The bubbles (7) are, in this case, facing the upper layer (1), to avoid the generation of marks on the viscera.

1. A protective dressing for abdominal viscera for application of negative pressure therapy in an open abdomen, made up of an upper layer and an intermediate layer integrally linked to each other at least by their respective perimeter edges, each of which consists of a laminar body, the protective dressing being characterized in that:
the upper layer includes a plurality of first through holes for the passage of fluids through the first through holes, and in that
the intermediate layer includes:
a plurality of bubbles filled with a low pressure gaseous fluid, and
a plurality of second through holes for the passage of fluids through the first through holes.

2. The protective dressing according to claim 1, characterized in that it includes a lower layer integrally linked to the intermediate layer, made up of a laminar body that includes the first through holes for the passage of fluids through the first through holes.

3. The protective dressing according to claim 2, characterized in that the upper layer, the lower layer, and the intermediate overlap each other so that the first through holes face each other with interposition of the intermediate layer.

4. The protective dressing according to claim 1, characterized in that two contiguous bubbles are separated from each other by a distance of at least half their diameter.

5. The protective dressing according to claim 2, characterized in that two contiguous bubbles are separated from each other by a distance of at least half their diameter.

6. The protective dressing according to claim 3, characterized in that two contiguous bubbles are separated from each other by a distance of at least half their diameter.
